(19) United States
(12) Patent Application Publication
(10) Pub. No.: US 2019/0328271 A1
(43) Pub. Date: Oct. 31, 2019

(54) Apparatus For Positioning A Head of A Neonate During An MRI

(71) Applicant: Aspect Imaging Ltd, Shoham (IL)

(72) Inventor: Itzhak Rabinovitz, Ness Tsiyona (IL)

(73) Assignee: Aspect Imaging Ltd, Shoham (IL)

(21) Appl. No.: 16/095,612

(22) PCT Filed: Apr. 6, 2017
(86) PCT No.: PCT/IL2017/050425

§ 371 Date: Oct. 22, 2018

Related U.S. Application Data
(60) Provisional application No. 62/325,241, filed on Apr. 20, 2016.

(51) Int. Cl. A61B 5/055; G01R 33/28

(52) U.S. Cl. CPC A61B 5/0555; A61B 2560/0437; A61B 2503/045; G01R 33/28

(57) ABSTRACT

A neonate positioning assembly for positioning a head of a neonate within a radio frequency coil of a magnetic resonance imaging is provided. The neonate positioning assembly allows for the head of the neonate to be positioned within a field of view of the radio frequency coil without repositioning the neonate on the positioning assembly.

### CROSS REFERENCE TO RELATED APPLICATIONS

This application claims priority to and the benefit of U.S. Provisional Patent Application No. 62/325,241, filed on Apr. 20, 2016, the entire contents of which are incorporated herein in reference in its entirety.

### FIELD OF THE INVENTION

The invention relates to the field of magnetic resonance imaging (MRI), and more particularly, to positioning a neonate for an MRI within an RF coil.

### BACKGROUND OF THE INVENTION

Magnetic Resonance Imaging (MRI) systems are becoming ever more popular in medical procedures involving medical images of patients. MRI systems can include magnets to generate a magnetic field and one or more RF coils to transmit and receive signals. In MRI systems, proximity of an RF coil to an object to be imaged (e.g., a head of a human) can effect a signal to noise ratio (SNR) of signals received by the MRI system.

Currently, when obtaining an MRI of a head of a neonate (e.g., human baby), the baby's head is positioned within an RF coil. Size of a neonate's head can vary greatly, such that one baby can have its head positioned substantially in the center of the coil, while another baby can have its head positioned substantially off center of the coil.

Having the baby's head substantially within the center of the coil can improve signal-to-noise ratio of the MRI images. Thus, when a baby has a head size such that its head is positioned substantially off center of the RF coil, currently pillows or other imprecise objects can be used to attempt to center the baby's head within the RF coil.

Therefore, it can be desirable to position a head of a baby substantially in a center of an RF coil with precision.

### SUMMARY OF THE INVENTION

In one aspect, the invention involves a neonate positioning assembly for positioning a head of a neonate within a radio frequency (RF) coil for magnetic resonance imaging (MRI) of the neonate. The neonate positioning assembly includes an elongated bed that the neonate lies upon during imaging, the elongated bed having a head portion for the head of the neonate at a first end of the elongated bed and a first pivot point at a second end of the elongated bed. The neonate positioning assembly also includes a base removably positioned under the elongated bed and having a first end and a second end, the second end of the base having a second pivot point that aligns with the first pivot point at the second end of the elongated bed such that the first end of the base is aligned with the first end of the elongated bed. The neonate positioning assembly also includes a fastener to fasten the elongated bed to the base via the first pivot point and the second pivot point. The neonate positioning assembly also includes a slider that slides along the base and the elongated bed between the fastener and the head portion such that when the slider is located at the fastener, elevation of the elongated bed is at a maximum, and as the slider slides away from the fastener, elevation of the elongated bed gradually decreases.

In some embodiments, the first pivot point and the second pivot point are openings and the fastener is a dowel. In some embodiments, the slider is maneuvered by manual mechanism, automatic mechanism, remote-controlled mechanism, semi-automatic mechanism and any combination thereof. In some embodiments, the bed, the base, the fastener and the slider are made of non-magnetic material.

### BRIEF DESCRIPTION OF THE DRAWINGS

Non-limiting examples of embodiments of the disclosure are described below with reference to figures attached hereto that are listed following this paragraph. Dimensions of features shown in the figures are chosen for convenience and clarity of presentation and are not necessarily shown to scale.

The subject matter regarded as the invention is particularly pointed out and distinctly claimed in the concluding portion of the specification. The invention, however, both as to organization and method of operation, together with objects, features and advantages thereof, can be understood by reference to the following detailed description when read with the accompanied drawings. Embodiments of the invention are illustrated by way of example and not limitation in the figures of the accompanying drawings, in which like reference numerals indicate corresponding, analogous or similar elements, and in which:

FIG. 1 is a diagram of a neonate positioning assembly, according to an illustrative embodiment of the invention.

FIG. 2 is a diagram of the neonate positioning assembly, according to an illustrative embodiment of the invention.

FIG. 3a is a diagram of a bed of the neonate positioning assembly of FIG. 2, according to an illustrative embodiment of the invention.

FIG. 3b is a diagram of a slider of the neonate positioning assembly of FIG. 2, according to an illustrative embodiment of the invention.

FIG. 3c is a diagram of a base of the neonate positioning assembly of FIG. 2, according to an illustrative embodiment of the invention.

FIG. 4 is a diagram of the neonate positioning assembly of FIG. 2 in an elevated position, according to an illustrative embodiment of the invention.

FIGS. 5A and 5B are diagrams of a MRI device that can receive a capsule having the neonate positioning assembly of FIG. 2, according to an illustrative embodiment of the invention.

FIG. 6 is a diagram of the capsule of FIG. 5B, according to an illustrative embodiment of the invention.

FIG. 7 is a diagram of the capsule of FIG. 5B, according to an illustrative embodiment of the invention.

### DETAILED DESCRIPTION OF THE INVENTION

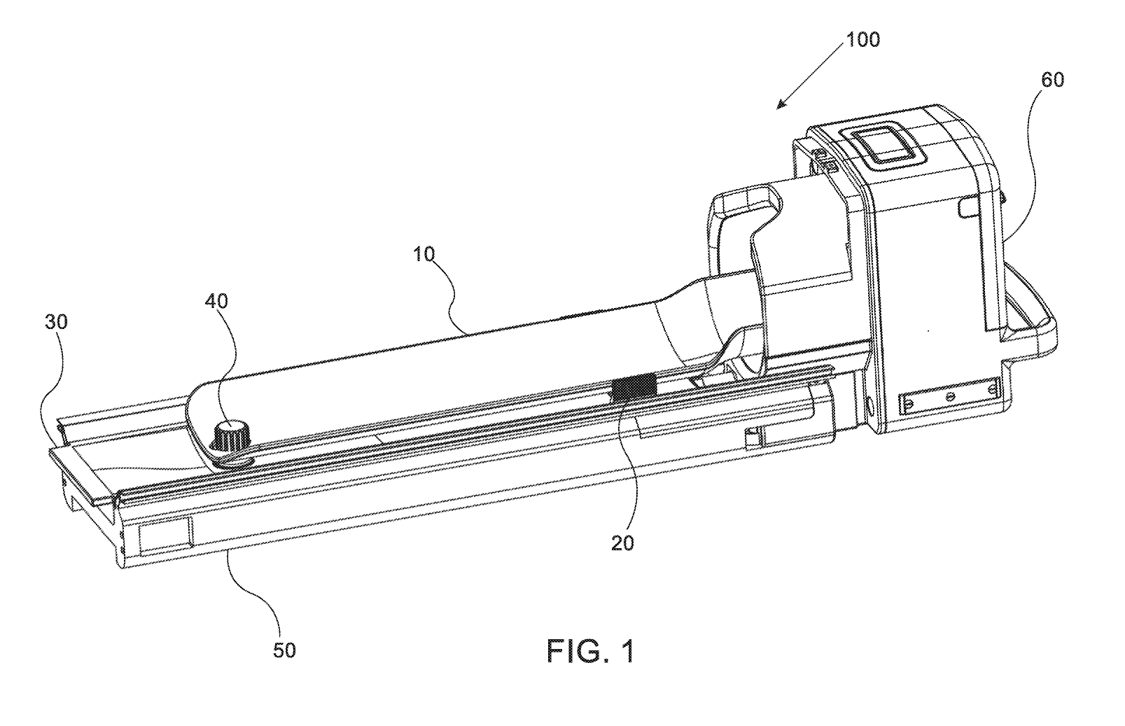

FIG. 1 is a diagram of a neonate positioning assembly 100, according to an illustrative embodiment of the invention. The neonate positioning assembly 100 includes a bed 10, a base 30, a fastener 40 and a slider 20. The neonate positioning assembly 100 can be positioned on a member 50 (e.g., a surface of an incubator and/or neonate transport capsule to position a neonate within a MRI device). The bed 10 can be partially inserted into an RF coil 60.

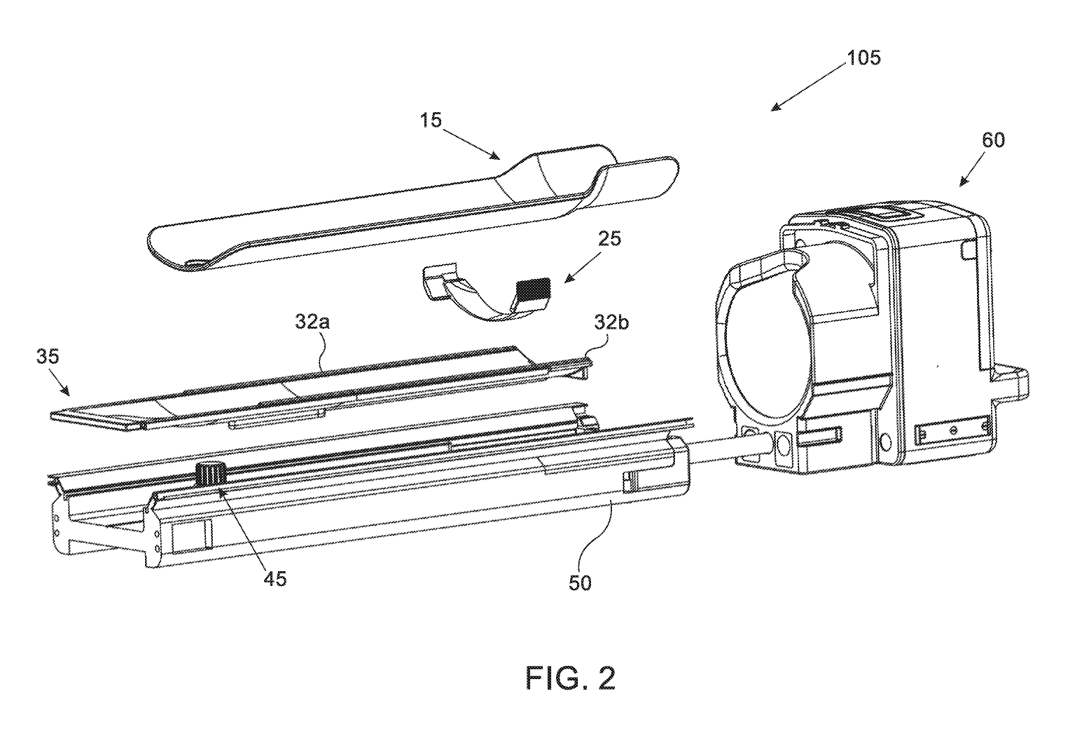

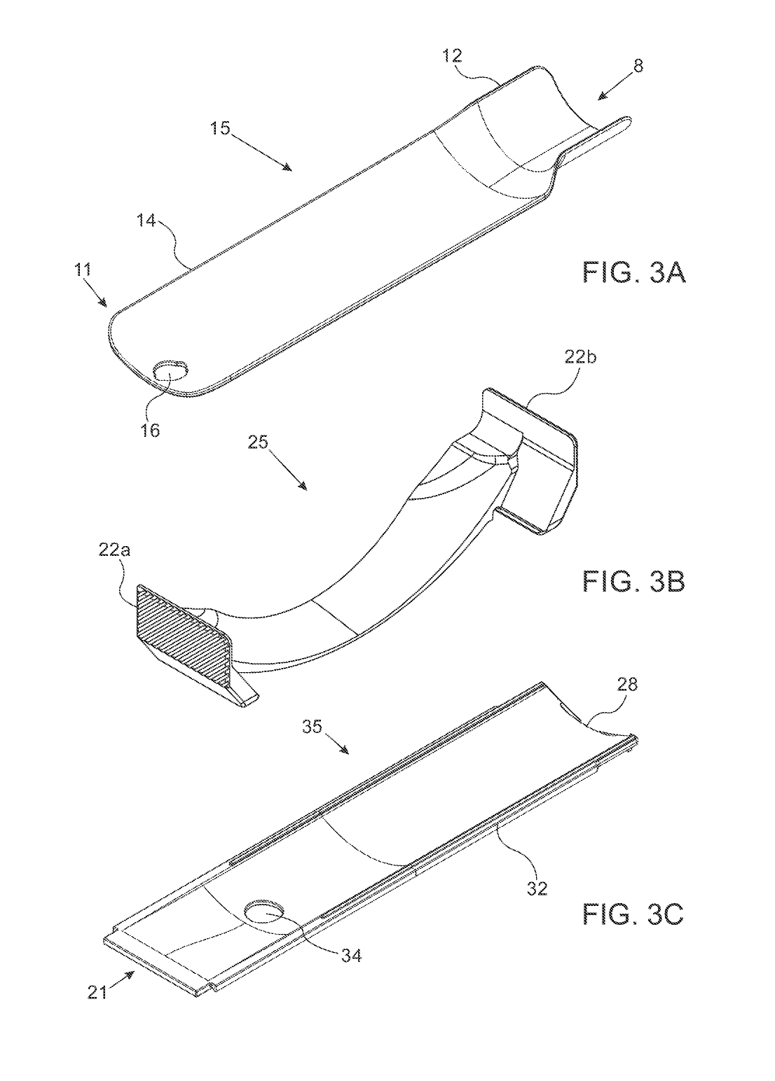

FIG. 2 is a diagram of the neonate positioning, according to an illustrative embodiment of the invention. The neonate positioning assembly 105 includes a bed 15, a base 35, a fastener 45 and a slider 25. The base 35 includes two grooved rails 32a and 32b and the slider 25 includes two members 22a and 22b (See FIG. 3B) that can slide in the two grooved rails 32a and 32b, respectively. The slider 25 can be curved.

FIG. 3a, FIG. 3b, and FIG. 3c are diagrams of the bed 15, the slider 25, and the base 35 of the neonate positioning assembly of FIG. 2, according to an illustrative embodiment of the invention. The bed 15 includes a head portion 12 at a first end 8 and a torso portion 14 at a second end 11. The head portion 12 is the portion that is inserted into an RF coil or RF coil assembly. The torso portion includes a first pivot point 16 (e.g., as shown an opening). The base 35 includes a first end 28 and a second end 21. The second end 21 includes a second pivot point 34 (e.g., as shown an opening). The bed 15 is positioned on top of the base 35 such that the first pivot point 16 and the second pivot point 34 align. A fastener (e.g., fastener 45 as described above) can secure the base 35 and the bed 15. The fastener 45 can be a dowel. The slider 25 is positioned between the bed 15 and the base 35 such that the members 22a and 22b are inserted into the two slides 32a and 32b, respectively. In this manner, the slider 25 can slide along the bed 15 to cause the head portion 12 of the bed to elevate.

The members 22a and 22b can be wheels, rack wheels, ball bearings, rollers, rolling discs, or any other mechanism as is known in the art for sliding. The sliders 32a and 32b can be a track, a guide, groove, rail, or any other mechanism as is known in the art for sliding. The slider 25 can be actuated via a manual mechanism, automatic mechanism, remote-controlled mechanism, semi-automatic mechanism or any combination thereof.

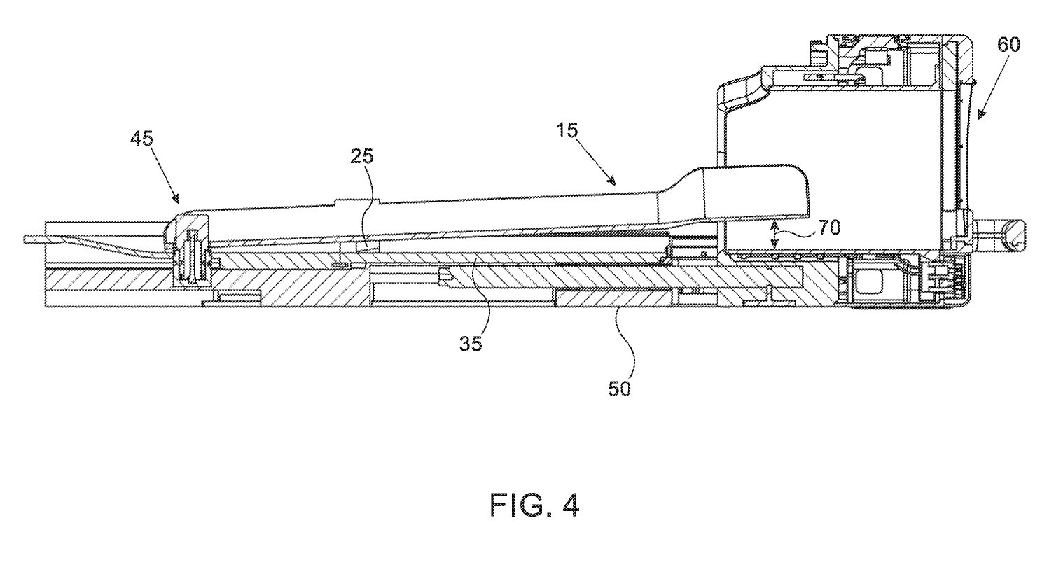

FIG. 4 is a diagram of the neonate positioning assembly of FIG. 2 in an elevated position, according to an illustrative embodiment of the invention. The slider 25 is positioned away from the fastener 45 causing an elevation 70 in the head portion of the bed 15. As the slider 25 slides further towards the fastener 45, the elevation 70 increases. As the slider 25 slides away from the fastener 45, the elevation 70 decreases. When a baby is lying on the bed 15 with its head in the head portion, elevating the head portion of the bed causes the baby's head to be repositioned within the RF coil 60. In this manner, a neonate's head can be particularly positioned within the RF coil 60. The position of the neonate's head can be aligned with the field of view (e.g., a particular position within the RF coil) without repositioning the neonate on the mattress while the neonate is within the RF coil.

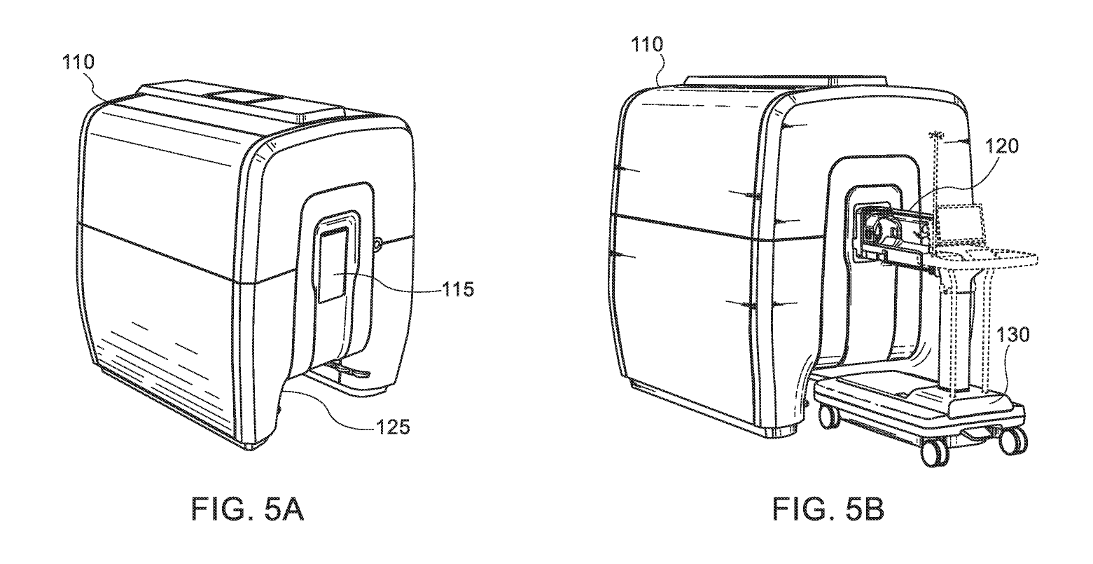

FIGS. 5A and 5B are diagrams of an MRI device 110 that can receive a neonate capsule 120 having the neonate positioning assembly of FIG. 2, according to an illustrative embodiment of the invention. The MRI device 110 can include a bore 115 and a recess 125. The bore 115 can receive the neonate capsule 120 and the recess 125 can receive a transport cart 130 that can be used to transport the neonate capsule 120, as shown in FIG. 5B. The neonate positioning assembly can be used inside of the neonate capsule 120. In this manner, an image of a neonate's head can be taken within the MRI, with the neonate's head in a particular position with the RF coil.

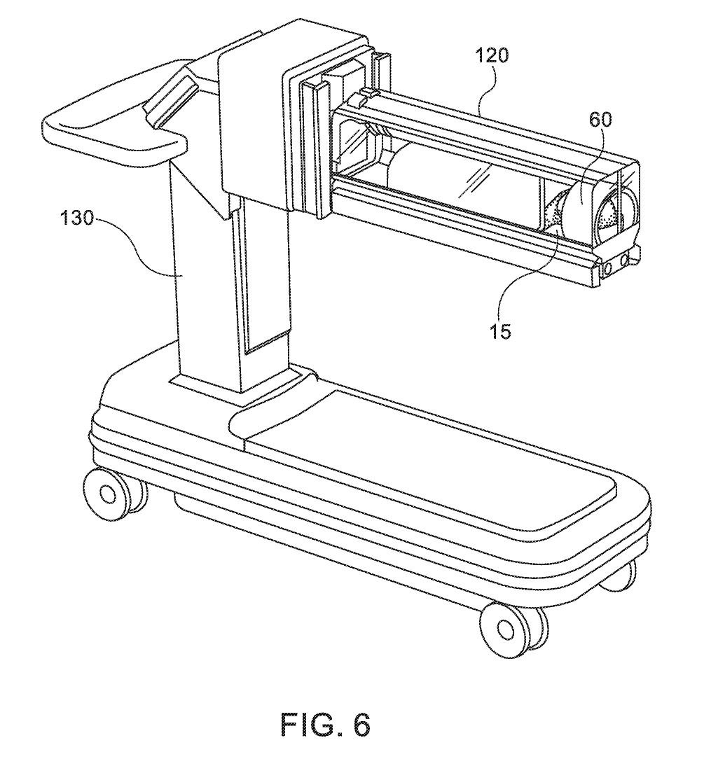

FIG. 6 is a diagram of a neonate capsule of FIG. 5B, according to an illustrative embodiment of the invention. The neonate is resting on the bed 15 of the neonate positioning assembly. The RF coil 60 is positioned around the neonate's head.

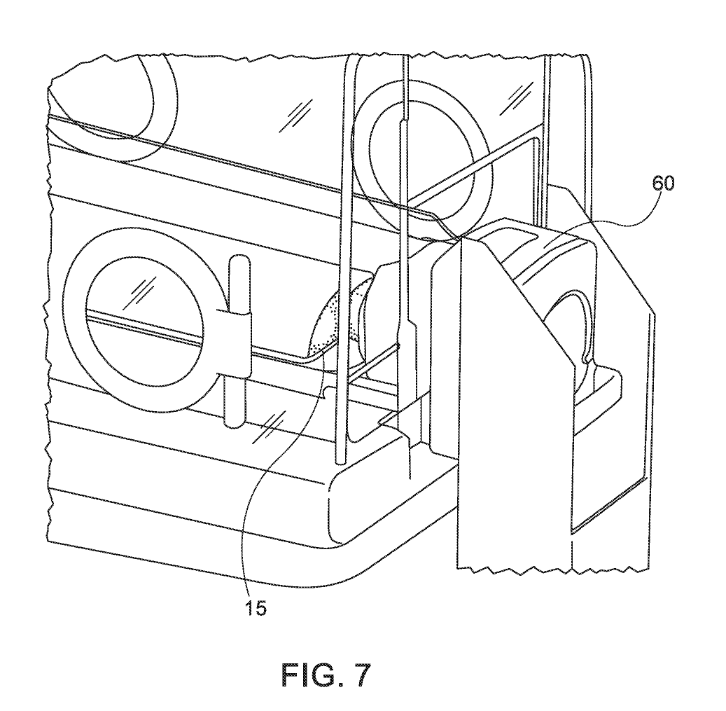

FIG. 7 is a diagram of the capsule of FIG. 5B, according to an illustrative embodiment of the invention. The neonate rests on the bed 15 which a RF coil positioning assembly 60 surround the head of the neonate.

1. A neonate positioning assembly for positioning a head of a neonate within a radio frequency (RF) coil for magnetic resonance imaging (MRI) of the neonate, the neonate positioning assembly comprising:
an elongated bed that the neonate lies upon during imaging, the elongated bed having a head portion for the head of the neonate at a first end of the elongated bed and a first pivot point at a second end of the elongated bed;
a base removably positioned under the elongated bed and having a first end and a second end, the second end of the base having a second pivot point that aligns with the first pivot point at the second end of the elongated bed such that the first end of the base is aligned with the first end of the elongated bed;
a fastener to fasten the elongated bed to the base via the first pivot point and the second pivot point; and
a slider that slides along the base and the elongated bed between the fastener and the head portion such that when the slider is located at the fastener, elevation of the elongated bed is at a maximum, and as the slider slides away from the fastener, elevation of the elongated bed gradually decreases.

2. The neonate positioning assembly of claim 1 wherein the first pivot point and the second pivot point are openings and the fastener is a dowel.

3. The neonate positioning assembly of claim 1 wherein the slider is maneuvered by manual mechanism, automatic mechanism, remote-controlled mechanism, semi-automatic mechanism and any combination thereof.

4. The neonate positioning assembly of claim 1 wherein the bed, the base, the fastener and the slider are non-magnetic material.
